(19) United States
(12) Patent Application Publication
(10) Pub. No.: US 2010/0267059 A1
(43) Pub. Date: Oct. 21, 2010

(54) IMMUNODEFICIENCY SCREENING AT POINT OF CARE

(76) Inventor: James W. Jacobson, Leander, TX (US)

Correspondence Address: FULBRIGHT & JAWORSKI L.L.P., 600 CONGRESS AVE., SUITE 2400, AUSTIN, TX, 78701, US

(21) Appl. No.: 12/763,865

(22) Filed: Apr. 20, 2010

Related U.S. Application Data
(60) Provisional application No. 61/170,984, filed on Apr. 20, 2009.

(51) Int. Cl. G01N 33/53

(52) U.S. Cl. 435/7.24

(57) ABSTRACT

Embodiments of the invention utilizes advanced detection methodologies as a cost-effective, efficient, ultra-sensitive rapid method for diagnosing severe combined immunodeficiency (SCID) in infants. In certain aspects, multiple markers of SCID are concurrently detected and measured to provide a more efficient, sensitive and accurate diagnosis of SCID.

This Application claims priority to U.S. Provisional Patent application Ser. No. 61/170,984 filed Apr. 20, 2009, which is incorporated herein by reference in its entirety.

### BACKGROUND OF THE INVENTION

1. Field of the Invention

The present invention relates generally to the fields of medicine, physiology, diagnostics, and biochemistry. In certain embodiments, the invention relates to microsieve-based assays to measure SCID-associated analytes.

2. Description of Related Art

Severe combined immunodeficiency diseases (SCIDs) represent a spectrum of disorders characterized by profound defects of both cellular and humoral immunity. One in every 10⁵ to 10⁶ live births is affected by these diseases. Infants with SCID usually become ill in the first few months of life. While their growth and development may initially proceed normally, infections leading to cessation of growth soon become evident. Individuals with SCID are vulnerable to virtually every type of pathogenic microorganism, even those that rarely cause disease in normal individuals. Candida fungal infection of mucocutaneous surfaces is often the first indication of immunodeficiency, followed by intractable diarrhea and pneumonia. The majority of infected infants die before their first birthday.

Classical SCID (“Swiss-type agammaglobulinemia”) is characterized by the absence of both T and B cells, presumably related to a defect affecting the lymphocytic stem cell. Autosomal recessive forms of SCID result from deficiencies of adenosine deaminase (ADA) or purine nucleoside phosphorylase (PNP), the inability to express class II molecules of the major histocompatibility complex (“Bare Lymphocyte Syndrome”), or defective IL-2 production. Other autosomal recessive forms have no known defect.

SCID is a rare disease that may be detected in newborn infants (i.e., birth to 1 year of age) by automated blood count and manual differential. Early diagnosis of Severe Combined Immunodeficiency (SCID) is critical—because chances for successful treatment are highest for infants who have not yet experienced severe opportunistic infections. Outcomes for infants with severe combined immunodeficiency (SCID) would be improved by universal newborn screening, but there are not yet screening tests of sufficient accuracy for the disorder. Therefore, there is a need to develop a more sensitive, accurate and cost-effective method for diagnosing SCID.

### SUMMARY OF THE INVENTION

Thus, in accordance with certain aspects of the present invention, there is provided a method for diagnosing severe combined immunodeficiency (SCID) in an infant comprising: (a) passing a blood sample from an infant through a flow cell comprising a microsieve that separates lymphocytes from the blood sample, wherein the lymphocytes are contacted with a fluorescence-emitting lymphocyte marker that binds CD4 and a fluorescence-emitting lymphocyte marker that binds CD 8, in combination with one or more of (i) a fluorescence-emitting lymphocyte marker that binds CD2, (ii) a fluorescence-emitting lymphocyte marker that binds CD19, or (iii) a fluorescence-emitting lymphocyte marker that binds CD56, forming a lymphocyte/marker complex; (b) exposing a lymphocyte-marker complex to light at a wavelength suitable for excitation of the fluorescence-emitting lymphocyte marker; (c) imaging fluorescence signals from the fluorescence-emitting lymphocyte marker to assess the number of lymphocytes in the sample; and (d) transforming the number of lymphocytes into a lymphocyte ratio and comparing the ratio with a selected reference, wherein a lower amount of the lymphocytes as compared to a normal control or about the same amount of the lymphocytes as compared to a SCID control is indicative of SCID in the subject.

Optionally the blood sample used in the methods taught herein is of a known volume. By “known volume” is meant a volume that is calculated or known prior to addition to the cartridge or a volume that can be calculated, measured, or metered once the sample is present in the flow cell.

In some embodiments the microsieve is a membrane. More particularly the microsieve may be a polycarbonate membrane. In a certain embodiment, analytes, particularly cells, including lymphocytes and other white blood cells, whose size is larger than the pores of the microsieve, are captured in the flow cell and immobilized on the membrane. The captured analytes may be analyzed directly or may be treated with visualization compounds prior to imaging.

In an embodiment, the blood sample may be a finger stick blood sample, a heel stick blood sample, or a venipuncture sample. Generally, the lymphocytes tested could be T cells, B cells or nature killer cells. In some embodiments, various sub-populations of specific cell types within a blood sample are distinguished. For example, the T cells present in a blood sample may be further categorized into helper (CD4⁺), cytotoxic (CD8⁺, memory (CD4/CD8 and/or CD45RO) or suppressor/regulatory (CD4⁺CD25⁺FOXP3⁺T cells. Alternatively, B cells present in a blood sample may be further categorized into populations of immature, mature, activated, memory, or plasma cells, based on the immunoglobulin isotype expressed on the cell surface, and presence or absence of various additional proteins.

In another aspect, the imaging may comprise using a CCD camera or a CMOS detector. A high sensitivity sensor array (e.g., CCD or CMOS) may be used to measure changes in optical characteristics which occur upon binding of the biological/chemical agents. A functional sensor array may be created by interfacing the flow cells with filters, light sources, fluid delivery and micromachined particle receptacles. In one embodiment, data acquisition and handling may be performed with existing CCD or CMOS technology. CCD or CMOS detectors may be configured to measure white light, ultraviolet light or fluorescence. Other detectors such as photomultiplier tubes, charge induction devices, photo diodes, photodiode arrays, and microchannel members may also be used.

In certain embodiments, the fluorescence-emitting marker may be coupled to a fluorescence-emitting particle, such as a particle with a red spectra, e.g., Alexa 647, or a particle with a green spectra, e.g., Alexa 488. Common fluorescence-emitting particle or fluorescent moieties also include fluorescein, cyanine dyes, coumarins, phycoerythrin, phycobiliproteins, dansyl chloride, TEXAS RED®, lanthanide complexes, and more ALEXAFLUOR® dyes (Invitrogen-Molecular Probes, Inc., Eugene, Oreg.). Derivatives of these compounds also are included as common fluorescent moieties.

In some further embodiments, the lymphocyte ratio could comprise the amount of a specific lymphocyte compared to the amount of total lymphocytes or the amount of a specific lymphocyte in a specific volume, for example, per microliter or per mililiter. The lymphocyte ratio can also be compared to an age-matched control group. In a further aspect, the selected reference may be a threshold value, wherein a lymphocyte ratio lower than the threshold value is indicative of SCID in the subject.

Embodiments discussed in the context of methods and/or compositions of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DESCRIPTION OF ILLUSTRATIVE EMBODIMENTS

The invention relates to microsieve-based assays to measure severe combined immunodeficiency (SCID)-associated analytes (e.g., T lymphocytes, B lymphocytes and nature killer (NK) cells) in subjects at risk for SCID. Methods and systems of the present invention are optimal for use in identifying SCID for newborns.

### I. Severe Combined Immunodeficiency (SCID)

In the present invention, there are provided methods and systems for identifying severe combined immunodeficiency (SCID) patients using a micosieve-based assay.

Severe combined immunodeficiency (SCID), or Boy in the Bubble Syndrome, is a genetic disorder in which both “arms” (B cells and T cells) of the adaptive immune system are crippled, due to a defect in one of several possible genes. SCID is a severe form of heritable immunodeficiency. It is also known as the “bubble boy” disease because its victims are extremely vulnerable to infectious diseases. Chronic diarrhea, ear infections, recurrent Pneumocystis jirovecii pneumonia, and profuse oral candidiasis commonly occur. These babies, if untreated, usually die within 1 year due to severe, recurrent infections. Classical SCID has a reported incidence of about 1 in 65,000 live births in Australia. Recent studies indicate that one in every 2,500 children in the Navajo population inherit severe combined immunodeficiency. This condition is a significant cause of illness and death among Navajo children. Ongoing research reveals a similar genetic pattern among the related Apache people.

Standard genetic testing for SCID is not currently available in newborns due to the diversity of the genetic defect. Some SCID can be detected by sequencing fetal DNA if a known history of the disease exists. Otherwise, SCID is not diagnosed until about six months of age, usually indicated by recurrent infections. The delay in detection is because newborns carry their mother's antibodies for the first few weeks of life and SCID babies look normal.

The most common treatment for SCID is bone marrow transplantation, which has been successful using either a matched related or unrelated donor, or a half-matched donor, who would be either parent. The half-matched type of transplant is called haploidentical and was perfected by Memorial Sloan Kettering Cancer Center in New York and also Duke University Medical Center which currently does the highest number of these transplants of any center in the world. Today, transplants done in the first three months of life have a high success rate. Physicians have also had some success with in utero transplants done before the child is born and also by using cord blood which is rich in stem cells.

More recently gene therapy has been attempted as an alternative to the bone marrow transplant. Transduction of the missing gene to hematopoietic stem cells using viral vectors is being tested in ADA SCID and X-linked SCID. The first gene therapy trials were performed in 1990, with peripheral T cells. In 2000, the first gene therapy “success” resulted in SCID patients with a functional immune system. These trials were stopped when it was discovered that two of ten patients in one trial had developed leukemia resulting from the insertion of the gene-carrying retrovirus near an oncogene. In 2007, four of the ten patients have developed leukemias. Work is now focusing on correcting the gene without triggering an oncogene. No leukemia cases have yet been seen in trials of ADA-SCID, which does not involve the gamma c gene that may be oncogenic when expressed by a retrovirus. Trial treatments of SCID have been gene therapy's only success; since 1999, gene therapy has restored the immune systems of at least 17 children with two forms (ADA-SCID and X-SCID) of the disorder.

All forms of SCID are inherited, with as many as half of SCID cases linked to the X chromosome, passed on by the mother. X-linked SCID results from a mutation in the interleukin 2 receptor gamma (IL2RG) gene which produces the common gamma chain subunit, a component of several IL receptors. IL2RG activates an important signalling molecule, JAK3. A mutation in JAK3, located on chromosome 19, can also result in SCID. Defective IL receptors and IL receptor pathways prevent the proper development of T-lymphocytes that play a key role in identifying invading agents as well as activating and regulating other cells of the immune system.

In another form of SCID, there is a lack of the enzyme adenosine deaminase (ADA), coded for by a gene on chromosome 20. This means that the substrates for this enzyme accumulate in cells. Immature lymphoid cells of the immune system are particularly sensitive to the toxic effects of these unused substrates, so fail to reach maturity. As a result, the immune system of the afflicted individual is severely compromised or completely lacking.

### II. Lymphocytes

In certain aspects of the invention, lymphocytes are detected and measured for diagnosis of SCID. A lymphocyte is a type of white blood cell in the vertebrate immune system. The three major types of lymphocyte are T cells, B cells and natural killer (NK) cells.

By their appearance under the light microscope, there are two broad categories of lymphocytes, namely the large granular lymphocytes and the small lymphocytes. Functionally distinct subsets of lymphocytes correlate with their appearance. Most, but not all large granular lymphocytes are more commonly known as the natural killer cells (NK cells). The small lymphocytes are the T cells and B cells. Lymphocytes play an important and integral role in the body's defenses.

T cells and B cells are the major cellular components of the adaptive immune response. T cells are involved in cell-mediated immunity whereas B cells are primarily responsible for humoral immunity (relating to antibodies). The function of T cells and B cells is to recognize specific “non-self” antigens, during a process known as antigen presentation. Once they have identified an invader, the cells generate specific responses that are tailored to maximally eliminate specific pathogens or pathogen infected cells. B cells respond to pathogens by producing large quantities of antibodies which then neutralize foreign objects like bacteria and viruses. In response to pathogens some T cells, called helper T cells produce cytokines that direct the immune response while other T cells, called cytotoxic T cells, produce toxic granules that induce the death of pathogen infected cells. Following activation, B cells and T cells leave a lasting legacy of the antigens they have encountered, in the form of memory cells. Throughout the lifetime of an animal these memory cells will “remember” each specific pathogen encountered, and are able to mount a strong response if the pathogen is detected again.

NK cells are a part of innate immune system and play a major role in defending the host from both tumors and virally infected cells. NK cells distinguish infected cells and tumors from normal and uninfected cells by recognizing alterations in levels of a surface molecule called MHC (major histocompatibility complex) class I. NK cells are activated in response to a family of cytokines called interferons. Activated NK cells release cytotoxic (cell-killing) granules which then destroy the altered cells.

NK-cells are defined as large granular lymphocytes that do not express T-cell antigen receptors (TCR) or Pan T marker CD3 or surface immunoglobulins (Ig) B cell receptor but that usually express the surface markers CD16 (FcγRIII) and CD56 in humans, and NK1.1/NK1.2 in certain strains of mice. Up to 80% of NK cells also express CD8.

It is impossible to distinguish between T cells and B cells in a peripheral blood smear. Normally, flow cytometry testing or other methods may be used for specific lymphocyte population counts based on typical recognition markers on cell surface (Table 1). This can be used to specifically determine the percentage of lymphocytes that contain a particular combination of specific cell surface proteins, such as immunoglobulins or cluster of differentiation (CD) markers or that produce particular proteins (for example, cytokines using intracellular cytokine staining (ICCS)). In order to study the function of a lymphocyte by virtue of the proteins it generates, other scientific techniques like the ELISPOT or secretion assay techniques can be used

TABLE 1
Typical recognition markers for lymphocytes

| LYMPHOCYTE | FUNCTION OF | | PHENOTYPIC |
|---|---|---|---|
| CLASS | LYMPHOCYTE | PROPORTION | MARKER(S) |
| NK cells | Lysis of virally | 7% (2-13%) | CD16, CD56, |
| | infected cells | | but not CD3 |
| | and tumour cells | | |
| Helper T | Release cytokines | 46% (28-59%) | TCRαβ, |
| cells | and growth | | CD3 and CD4 |
| | factors that | | |
| | regulate other | | |
| | immune cells | | |
| Cytotoxic T | Lysis of virally | 19% (13-32%) | TCRαβ, |
| cells | infected cells, | | CD3 and CD8 |
| | tumour cells | | |
| | and allografts | | |
| γδ T | Immunoreg- | | TCRγδ |
| cells | ulation and | | and CD3 |
| | cytotoxicity | | |
| B cells | Secretion of | 23% (18-47%) | MHC class II, |
| | antibodies | | CD19 and CD21 |

### III. Cell Surface Antigens

In some embodiments, the cellular components of a sample may be characterized by detecting the presence and/or expression levels of one more molecular groups (e.g., polypeptides, polynucleotides, carbohydrates, lipids) typically known to be associated or correlated with a specific trait for which the test is being performed. For example, a blood sample may be collected to measure the number of one or more specific cell types present in the sample (commonly referred to in the art as “cell counts”), and/or the ratio thereof with respect to one or more different cells types also present in the sample. Examples of the types of blood cells that may be detected in a blood sample include, but are not limited to, erythrocytes, lymphocytes (e.g., T cells and B cells), Natural Killer (NK)-cells, monocytes/macrophages, megakaryocytes, platelets, eosinophils, neutrophils, basophils or mast cells, in some embodiments, various sub-populations of specific cell types within a fluid sample are distinguished. For example, the T cells present in a blood sample may be further categorized into helper (CD4⁺), cytotoxic (CD8⁺), memory (CD4/CD8 and/or CD45RO) or suppressor/regulatory (CD4⁺CD25⁺FOXP3⁺) T cells. Alternatively, B cells present in a blood sample may be further categorized into populations of immature, mature, activated, memory, or plasma cells, based on the immunoglobulin isotype expressed on the cell surface, and presence or absence of various additional proteins. It should be understood, that the presently disclosed systems and methods may be suitably adapted to analyze most cell types and/or macromolecules present in a biological sample without departing from the spirit and scope of the presently described embodiments.

Analysis of a cellular composition of a sample may include detecting the presence of one or more “surface markers” known to be expressed on the surface of the population of cells of interest. Certain surface markers useful in the differential identification of cells in a sample (e.g., in particular cells involved in immune responses) and/or diseases are commonly referred to as “cluster of differentiation (CD)” antigens or CD markers, of which over 250 have been characterized. Many of the CD antigens may also be referred to by one or more alternative art-recognized terms. Table 2 lists several examples of CD antigens, and the cells in which they are expressed, that may be referred to using one or more alternative terms. The system of CD marker nomenclature is widely recognized by ordinary practitioners of the art. General guidance in the system of CD marker nomenclature, and the CD expression profiles of various cells may be found in most general immunology reference textbooks such as, for example, in IMMUNOLOGY, 4th Edition Ed. Roitt, Brostoff and Male chapter 28 and Appendix II (Mosby/Times Mirror International Publication 1998), or in IMMUNOBIOLOGY: THE IMMUNE SYSTEM IN HEALTH AND DISEASE, 5th Edition, Eds. Janeway et al. Appendices I-IV (Garland Publishing, Inc. 2001).

TABLE 2
Examples of CD antigens

| CD | | |
|---|---|---|
| Antigen | Identity/function | Expression |
| CD2 | T cell adhesion | T cells, NK cells |
| | molecule | |
| CD3 | T cell receptor | Thymocytes, T cells |
| | (γ, δ, ε, ξ, η) | |
| CD4 | MHC class II receptor | Thymocyte subsets, T helper |
| | | cells, monocytes, macrophages |
| CD8 | MHC class I receptor | Thymocyte subsets, |
| | | cytotoxic T cells |
| CD10 | Neutral | T and B cell precursors, |
| | endopeptidase/CAALA | activated B cells, granulocytes |
| CD11a | Integrin α | Lymphocytes, granulocytes, |
| | | monocytes and macrophages |
| CD11b | Integrin α | Myeloid and NK cells |
| CD13 | Aminopeptidase N | Granulocytes, monocytes |
| CD16 | FcγRIIIA/B | Neutrophils, NK cells, |
| | | macrophages |
| CD19 | B cell function/ | B cells |
| | activation | |
| CD20 | Ca²⁺ ion channel | B cells |
| CD21 | C3d and EBV receptor | Mature B cells |
| CD35 | Complement | Erythrocytes, B cells, monocytes, |
| | receptor 1 | neutrophils, eosinophils |
| CD41 | αIIb integrin | Platelets, megakaryocytes |
| CD45RO | Fibronectin | T cell subsets, B cell subsets, |
| | type II | monocytes, macrophages |
| CD45RA | Fibronectin | B cell, T cell subsets, |
| | type II | (naïve T cells), monocytes. |
| CD45RB | Fibronectin | T cell subsets, B cells, monocytes, |
| | type II | macrophages, granulocytes |
| CD56 | NKH-1 | NK cells |

In particular embodiments, one or more lymphocyte markers will be used for diagnosis and prognosis of SCID in infants, such as CD4, CD8, CD2, CD19 and CD56.

CD4 (cluster of differentiation 4) is a glycoprotein expressed on the surface of T helper cells, regulatory T cells, monocytes, macrophages, and dendritic cells. In humans, the CD4 protein is encoded by the CD4 gene. CD4 is a co-receptor that assists the T cell receptor (TCR) to activate its T cell following an interaction with an antigen presenting cell. Using its portion that resides inside the T cell, CD4 amplifies the signal generated by the TCR by recruiting an enzyme, known as the tyrosine kinase lck, which is essential for activating many molecules involved in the signaling cascade of an activated T cell. CD4 also interacts directly with MHC class II molecules on the surface of the antigen presenting cell using its extracellular domain.

CD8 (cluster of differentiation 8) is a transmembrane glycoprotein that serves as a co-receptor for the T cell receptor (TCR). Like the TCR, CD8 binds to a major histocompatibility complex (MHC) molecule, but is specific for the class I MHC protein. [2] There are two isoforms of the protein, alpha and beta, each encoded by a different gene. In humans, both genes are located on chromosome 2 in position 2p12. The CD8 co-receptor is predominantly expressed on the surface of cytotoxic T cells, but can also be found on natural killer cells.

CD2 (cluster of differentiation 2) is a cell adhesion molecule found on the surface of T cells and natural killer (NK) cells. It has also been called T-cell surface antigen T11/Leu-5, LFA-2, LFA-3 receptor, erythrocyte receptor and rosette receptor. It interacts with other adhesion molecules, such as lymphocyte function-associated antigen-3 (LFA-3/CD58) in humans, or CD48 in rodents, which are expressed on the surfaces of other cells. In addition to its adhesive properties, CD2 also acts as a co-stimulatory molecule on T and NK cells.

CD19 (Cluster of Differentiation 19), is a human protein encoded by the CD19 gene. Lymphocytes proliferate and differentiate in response to various concentrations of different antigens. The ability of the B cell to respond in a specific, yet sensitive manner to the various antigens is achieved with the use of low-affinity antigen receptors. This gene encodes a cell surface molecule which assembles with the antigen receptor of B lymphocytes in order to decrease the threshold for antigen receptor-dependent stimulation. CD19 is expressed on follicular dendritic cells and B cells. In fact, it is present on B cells from earliest recognizable B-lineage cells during development to B-cell blasts but is lost on maturation to plasma cells. It primarily acts as a B cell co-receptor in conjunction with CD21 and CD81. Upon activation, the cytoplasmic tail of CD19 becomes phosphorylated which leads to binding by Src-family kinases and recruitment of PI-3 kinase.

CD56 (Cluster of differentiation 56, or Neural Cell Adhesion Molecule (NCAM)) is a homophilic binding glycoprotein expressed on the surface of neurons, glia, skeletal muscle and natural killer cells. CD56 or NCAM has been implicated as having a role in cell-cell adhesion, neurite outgrowth, synaptic plasticity, and learning and memory. Normal cells that stain positively for CD56 include NK cells, activated T cells, the brain and cerebellum, and neuroendocrine tissues.

### IV. Antibodies

The analyte detection systems and methods may include, for example, various receptor molecules (such as specific antibodies) that bind to cell surface markers (e.g., CD markers or other disease-associated molecules) or any other analyte suspected to be present in a sample that allows rapid characterization of the sample. In some embodiments, one or more antibodies (e.g., monoclonal and/or polyclonal antibodies) that specifically recognize and bind to macromolecules expressed on the surface of cells (e.g., CD or other cell surface markers) may be used in an analyte detection system.

While certain specific examples of monoclonal or polyclonal antibodies are set forth above, it will be readily understood by ordinary practitioners of the art that the presently described analyte detection systems may be used, without limitation, in conjunction with any type of antibody that recognizes any antigen, including, but not limited to, commercially available antibodies or antibodies generated specifically for the purpose of performing the tests described herein. Monoclonal and polyclonal antibody design, production and characterization are well-developed arts, and the methods used therein are widely known to ordinary practitioners of the art (see, e.g., “Antibodies: A Laboratory Manual,” E. Howell and D. Lane, Cold Spring Harbor Laboratory, 1988). For example, a polyclonal antibody is prepared by immunizing an animal with an immunologically active composition including at least a portion of the macromolecule to which the desired antibody will be raised and collecting antiserum from that immunized animal. A wide range of animal species may be used for the production of antiserum. Examples of animals used for production of polyclonal anti-sera are rabbits, mice, rats, hamsters, horses, chickens, or guinea pigs.

A monoclonal antibody specific for a particular macromolecule can be readily prepared through use of well-known techniques such as those exemplified in U.S. Pat. No. 4,196,265, to Koprowski et al., which is herein incorporated by reference. Typically, the technique involves first immunizing a suitable animal with a selected antigen (e.g., at least a portion of the macromolecule against which the desired antibody is to be raised) in a manner sufficient to provide an immune response. Rodents such as mice and rats are preferred species for the generation of monoclonal antibodies. An appropriate time after the animal is immunized, spleen cells from the animal are harvested and fused, in culture, with an immortalized myeloma cell line.

The fused spleen/myeloma cells (referred to as “hybridomas”) are cultured in a selective culture medium that preferentially allows the survival of fused splenocytes. After the fused cells are separated from the mixture of non-fused parental cells, populations of B cell hybridomas are cultured by serial dilution into single-clones in microtiter plates, followed by testing the individual clonal supernatants for reactivity with the immunogen. The selected clones may then be propagated indefinitely to provide the monoclonal antibody of interest. In some embodiments, a microsieve-based detection system for use in performing WBC counts on a blood sample may use one or more polyclonal or monoclonal antibodies that specifically recognize various cell types that constitute WBCs to visualize specific blood cells. Antibodies suitable for this purpose include, but are not limited to: anti-CD3; anti-CD4; anti-CD8; anti-CD 16; anti-CD56; and/or anti-CD19 antibodies to specifically recognize: T cells; T helper cells and monocytes/macrophages; cytotoxic T cells; neutrophils, NK cells and macrophages; NK cells; and B cells, respectively.

Also useful as a binding agent in the system taught herein are chimeric antibodies and hybrid antibodies, with dual or multiple antigen or epitope specificities, and fragments, such as F(ab′)2, Fab′, Fab, Fv and the like, including hybrid fragments. Such binding agents retain their ability to bind their specific antigens. For example, fragments of antibodies which maintain CD4-binding activity are included within the meaning of the term “CD4 antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also useful herein are conjugates of antibody fragments and antigen binding proteins (single chain antibodies) as described, for example, in U.S. Pat. No. 4,704,692, the contents of which are hereby incorporated by reference. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. These Fvs lack the constant regions (Fc) present in the heavy and light chains of the native antibody.

### V. Detectable Labels

In certain aspects, the lymphocyte marker that binds lymphocytes may be coupled to a detectable label, such as a fluorescent agent to produce a fluorescence-emitting marker.

As used herein, the term “detectable label” is intended to mean any suitable agent, such as a chemical agent, that interacts with binding agents and allows the visualization of analyte/binding agent complexes. Detectable labels include, but are not limited to, enzymes, fluorophores, biotin, chromophores, radioisotopes, colored particles, electrochemical, chemical-modifying or chemiluminescent moieties.

The detection of the detectable label can be direct provided that the detectable label is conjugated to the binding agent of the system. Alternatively, the detection of the detectable label can be indirect. Thus, in some embodiments, a detectable label may bind indirectly to a binding agent by binding to a secondary agent that binds to the binding agent. Examples of secondary agents may include, but are not limited to DNA, RNA, proteins, enzymes, oligopeptides, oligonucleotides, antigens, and antibodies. In some embodiments, the secondary agent may be a polypeptide molecule that binds to a receptor or cell surface molecule. Alternatively, the secondary agent may include a secondary antibody directed against a receptor or cell surface molecule. In some embodiments, a method of detecting multiple analytes in a fluid may rely on immunological reactions that take place on the surface of the cells. In some cases tertiary or additional agents may be used. For example, a secondary or tertiary antibody may be coupled to the detectable label and the secondary or tertiary label would then be amplified as compared to a direct detection method.

“Detectable label is used interchangeably herein with “stain” or “label,” and a “stained” or “labeled” cell refers to a cell that is bound directly or indirectly to a detectable label.

Detectable labels include fluorescent microspheres or beads. Microspheres may be labeled with two or more fluorochromes mixed together in varying concentrations, such that each specific label has a specific concentration of each fluorochrome. It is the specific concentrations of the various fluorochromes together to provide a spectrum of labels that can be used to distinguish the various subsets of labeled microspheres. Thus, microspheres having detectably different labels may comprise the detectable labels used herein. See, e.g., WO99/19515 and WO 99/37814, which are incorporated herein in their entirety for types of microspheres and methods of making and using same. For example, the microspheres can be polystyrene-divinylbenzene microspheres or latex microparticles (available, for example, from Invitrogen.).

In some embodiments, at least one of the detectable labels is a fluorophore or a fluorescent microparticle (e.g., microsphere or bead). As used herein, the terms “fluorochrome” and “fluorphore” and the terms “microsphere’ and “microparticle” are used interchangeably. In some embodiments, a detectable label includes a fluorescent moiety.

A fluorescent agent, or a fluorophore, in analogy to a chromophore, is a component of a molecule which causes a molecule to be fluorescent. It is a functional group in a molecule which will absorb energy of a specific wavelength and re-emit energy at a different (but equally specific) wavelength. The amount and wavelength of the emitted energy depend on both the fluorophore and the chemical environment of the fluorophore. Common fluorescent moieties include fluorescein, cyanine dyes, coumarins, phycoerythrin, phycobiliproteins, dansyl chloride, TEXAS RED® and ALEXAFLUOR® dyes (Invitrogen-Molecular Probes, Inc., Eugene, Oreg.) and lanthanide complexes. Derivatives of these compounds also are included as common fluorescent moieties. The spectra of common fluorophores can be found at http://info.med.yale.edu/genetics/ward/tavi/FISHdyes2.html.

Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores such as the Alexa Fluors and the DyLight Fluors are generally more photostable, brighter, and less pH-sensitive than other standard dyes of comparable excitation and emission

For example, Alexa 350, Alexa 430, Alexa 488, Alexa 532, Alexa 546, Alexa 568, and Alexa 594 dyes are a new series of fluorescent dyes with emission/excitation spectra similar to those of AMCA, Lucifer Yellow, fluorescein, rhodamine 6G, tetramethylrhodamine or Cy3, lissamine rhodamine B, and Texas Red, respectively (the numbers in the Alexa names indicate the approximate excitation wavelength maximum in nm). All Alexa dyes and their conjugates are more fluorescent and more photostable than their commonly used spectral analogues listed above. In addition, Alexa dyes are insensitive to pH in the 4-10 range.

### VI. Detection Systems and Methods

In certain aspects of the invention, membrane-based systems and methods may be used to detect signals from lymphocyte markers to measure lymphocytes in a blood sample as described below.

A. Analyte Detection Systems

Details regarding analyte detection systems can be found in the following U.S. patents and patent applications, all of which are incorporated herein by reference in their entirety for the systems taught therein: U.S. Pat. No. 6,906,770 entitled “Fluid Based Analysis of Multiple Analytes by a Sensor Array”; U.S. Pat. No. 6,680,206 entitled “Sensor Arrays for the Measurement and Identification of Multiple Analytes in Solutions”; U.S. Pat. No. 6,602,702 entitled “Detection System Based on an Analyte Reactive Particle”; U.S. Pat. No. 6,589,779 entitled “General Signaling Protocols for Chemical Receptors in Immobilized Matrices”; U.S. patent application Ser. No. 09/616,731 entitled “Method and Apparatus for the Delivery of Samples to a Chemical Sensor Array”; U.S. patent application Ser. No. 09/775,342 entitled “Magnetic-Based Placement and Retention of Sensor Elements in a Sensor Array” (Published as U.S. Publication No.: 2002-0160363-A1); U.S. patent application Ser. No. 09/775,340 entitled “Method and System for Collecting and Transmitting Chemical Information” (Published as U.S. Publication No.: 2002-0064422-A1); U.S. patent application Ser. No. 09/775,344 entitled “System and Method for the Analysis of Bodily Fluids” (Published as U.S. Publication No.: 2004-0053322); U.S. Pat. No. 6,649,403 entitled “Method of Preparing a Sensor Array”; U.S. patent application Ser. No. 09/775,048 entitled “System for Transferring Fluid Samples Through A Sensor Array” (Published as U.S. Publication No.: 2002-0045272-A1); U.S. patent application Ser. No. 09/775,343 entitled “Portable Sensor Array System” (Published as U.S. Publication No.: 2003-0186228-A1); U.S. patent application Ser. No. 10/072,800 entitled “Method and Apparatus for the Confinement of Materials in a Micromacliined Chemical Sensor Array” (Published as U.S. Publication No.: 2002-0197622-A1); and U.S. patent application Ser. No. 10/427,744 entitled “Method and System for the Detection of Cardiac Risk Factors” (Published as U.S. Publication No.: 2004-0029259.

Further details regarding microsieve- or membrane-based detection systems can be found in the following U.S. Provisional Applications and PCT Applications, all of which are hereby incorporated herein by reference in their entirety for the systems taught therein: U.S. Provisional Application No. 60/736,082, entitled “Analyte Detection Systems and Methods Including Self-Contained Cartridges with Detection Systems and Fluid Delivery Systems,” filed on Nov. 10, 2005; PCT Application No. PCT/US05/06074 (WO 05/085796) entitled “Integration of Fluids and Reagents into Self-Contained Cartridges Containing Sensor Elements,” filed Feb. 28, 2005; PCT Application No. PCT/US05/06350 (WO 05/085855) entitled “Integration of Fluids and Reagents into Self-Contained Cartridges Containing Sensor Elements and Reagent Delivery Systems,” filed Feb. 28, 2005; PCT Application No. PCT/US05/06349 (WO 05/083423) entitled “Integration of Fluids and Reagents into Self-85589370.1 Contained Cartridges Containing Particle Based Sensor Elements and Membrane-Based Sensor Elements,” filed Feb. 28, 2005; PCT Application No. PCT/US05/06077 (WO 05/085854) entitled “Particle on Membrane Assay System,” filed Feb. 28, 2005; and PCT Application No. PCT/US05/06593 (WO 05/090983) entitled “Membrane Assay System Including Preloaded Particles,” filed Feb. 28, 2005.

In some embodiments, the system may include one or more disposable cartridges. A disposable sample cartridge may be the chemical and biochemical-sensing component of the analysis instrument. A cartridge may include index-matching, molded or machined plastics, metals, glass or a combination thereof. A cartridge may also include one or more reservoirs for holding reagents, samples, and/or waste. Reservoirs may be coupled to a cartridge via one or more microfluidic channels.

A cartridge may include one or more detection systems. As used herein the term “detection system” refers to a system having an analyte detection platform (e.g., a microsieve-based analyte detection platform). In some embodiments, a cartridge may be designed such that the cartridge is removably positionable in an instrument. Cartridge alignment may be performed manually or automatically using the cartridge positioning system. A cartridge positioning system may automatically or manually position the disposable cartridge in the instrument. In certain embodiments, the disposable cartridge may be placed in the cartridge self-positioning system prior to sample introduction. In one embodiment, a fluid delivery system may deliver reagents to a disposable cartridge. Once the disposable cartridge is placed inside the instrument, the cartridge positioning system may be used to align the one or more areas of the cartridge containing the sample to be analyzed with the instrument's optical platform. The optical platform may acquire images (e.g., visual or fluorescent) of the sample. The images may be processed and analyzed using software, algorithms, and/or neural networks.

Provided herein in certain aspects is a cartridge for differential assay of white blood cell populations. The cartridge comprises a chamber; a microsieve (e.g., a membrane) positioned at least partially within the chamber, wherein pores of the microsieve are configured to retain white blood cells from a blood sample and to allow red blood cells to pass through the microsieve, and wherein an image can be obtained from the microsieve; three or more binding agents contained at least partially in or on the cartridge, wherein each binding agent differentially binds one or more populations of white blood cells; and two or more detectable labels contained at least partially in or on the cartridge, wherein at least one of the detectable labels binds at least one of the binding agents.

The cartridge may be positioned, automatically or manually, in a housing of a analyte detection system. The cartridge may substantially contain all fluids used for the analysis.

In some embodiments, a check of the cartridge may be performed. For example, the cartridge includes one or more detectable labels to be determined. An image of the label may be obtained by one of the detectors. Analysis of the image is performed to determine if the known analyte can be detected. If the known analyte is detected, the cartridge is deemed suitable for use. If the known analyte is not detected, the cartridge may be disposed of and a new cartridge obtained, hi some embodiments, the new cartridge is obtained from the kit or a supply of cartridges.

At least a portion of the sample may be provided to a metered volume portion of the cartridge, in some embodiments, the sample may be drawn by capillary action into the metered volume portion, in certain embodiments, the sample may be delivered by a fluid delivery system disposed in or coupled to the cartridge. After the sample has filled the metered volume portion, a portion of the sample may travel toward an overflow reservoir, in some embodiments, the sample may not be measured.

B. Fluid Delivery

A fluid delivery system that includes a reagent may be actuated. Flow of fluid from the fluid delivery system may push a metered volume of sample from the metered volume portion towards a detection region that includes a microsieve-based detection system. The reagent and sample may combine during passage of the sample toward the one or more detection regions to form a sample/reagent mixture. A portion of the sample/reagent mixture flows through or is collected in the detection region. The remaining portion of sample/reagent mixture may flow over or through the detection region to a waste region of the cartridge.

In some embodiments, the fluid delivery system is not necessary to push the sample towards the detection region. Capillary forces may transport the sample towards the detection region. In some embodiments, capillary forces that transport the sample are enhanced with hydrophilic materials (e.g., plastic or glass) to coat a channel for aqueous samples. Certain portion of channels may include hydrophilic materials positioned proximate the collection region, in the metered volume chamber, and/or proximate the overflow reservoir to direct flow of aqueous samples through a cartridge.

In some embodiments, the sample may be drawn into a channel via negative pressure in the channel. For example, suction created by a passive valve or a negative pressure source may create negative pressure in a portion of a channel and draw fluids towards the detection region. In some embodiments, valves may be used to direct the flow of fluid and/or sample through the cartridge.

One or more additional fluid delivery systems may be actuated to release one or more additional fluids (e.g., additional PBS, water, or other buffers). One or more of the additional fluids may flow over or through one or more reagent regions (e.g., a reagent pad or through a channel containing reagents). One or more reagents (e.g., one or more antibodies or a detectable label) in or on the reagent regions may be reconstituted by the additional fluids. The reconstituted reagents may be transported to the detection region of the cartridge. Transport of the reconstituted reagents may be accomplished by continued actuation of the fluid delivery systems or through other methods described herein. The reconstituted reagents may label and wash a portion of the sample collected in one or more detection regions of the cartridge (e.g., wash WBCs retained on a microsieve).

Portions of a sample and/or fluids may be provided to a detection region in a cartridge sequentially, successively, or substantially simultaneously. In some embodiments, a portion of the sample moves towards a detection region as a portion of the fluid from the second fluid delivery system flows towards a reagent region. Fluid from the second fluid delivery system may reconstitute and/or collect one or more reagents from the reagent region and deliver the reagents to the detection region after the sample has passed through the detection region. The collected reagents may then be added to an analytes that have been collected by the detection region.

Valves (e.g., pinch valves, active valves, passive valves) and/or vents may be use to regulate flow of the sample. For example, a valve proximate the collection region may inhibit additional sample from flowing towards the detection region, hi some embodiments, one or more changes in elevation of a channel may inhibit the sample form entering other channels.

In some embodiments, a reagent (e.g., a detectable label or one or more antibodies) may be directly added to the matter on a microsieve of a microsieve-based detection system. The sample may then be washed with fluid remaining in the first fluid delivery system or with the fluid from one or more of the fluid delivery systems.

In some embodiments, only one fluid delivery system is used. For example, one or more syringes may be at least partially coupled to, positioned in, or positioned on the cartridge. Each syringe may contain one or more fluids to be used during the analysis. The syringes may be actuated and the fluids delivered sequentially, successively, or substantially simultaneously to the collection region, the reagent regions and/or the detection region.

In some embodiments, analytes collected on a microsieve of a microsieve-detection system may be viewed through a viewing chamber of the microsieve-detection system. Light sources may be activated and light may be directed towards the microsieve-based detection system. Light may enter the microsieve-detection system through a viewing chamber and/or a top layer of the microsieve-detection system. A detector may collect a signal produced from interaction of light with one or more analytes in the detection region, hi some embodiments, the detector may be optically aligned with the viewing chamber of the microsieve to allow the microsieve and/or detection region to be viewed by detector.

The detector processes the produced signal to produce images representative of the analytes collected by the detection system. Images may be obtained concurrently or simultaneously. Images may be analyzed and the analytes in the sample assessed.

The cartridge may then be removed from the analyzer and discarded. The above-described method may then be repeated for the next sample. In certain embodiments, portions of the analyzer may be disinfected between samples. In some embodiments, the cartridge is self-contained such that all fluids remain in the cartridge and the analyzer may not need to be disinfected.

Interaction of a sample with light produces a signal that is received by the detector. The detector may produce images from the signal. Images may be analyzed by an analyzer (e.g., automatically with a computer or manually by a human) to determine the analytes present in the sample.

A third fluid delivery system may be activated to allow a wash solution to flow through or over the detection region. The detection region may be washed repeatedly to clear the detection region and prepare for additional use.

The first fluid delivery system may be actuated, or a fourth fluid delivery system may be used, to push a second portion of sample towards the microsieve. The analysis may be repeated to determine different and/or duplicate sample analysis.

The procedure may be repeated as necessary to obtain the needed data. Additional samples may also be obtained and used. In some embodiments, one or more microsieves may be used in a microsieve-based detection system. After all analyses have been completed, the cartridge may be properly discarded.

In certain embodiments, a fluid delivery system may include metered pumps (e.g., syringe, rotary, and/or peristaltic), valves, connectors, and/or pressure-driven actuation (e.g., roller with motorized translation). A fluid delivery system may be vacuum-driven (e.g., a cartridge may be under vacuum). A fluid delivery system may draw one or more samples into an instrument, deliver one or more samples to a sample cartridge, and/or move fluids such as sample, reagents and/or buffers through the cartridge and other channels or fluid lines. A fluid delivery system may deliver samples and/or other fluids to a waste reservoir after analysis, hi one embodiment, a fluid delivery system may be used to wash a cartridge after sample analysis. Fluid may be driven through a cartridge after a sample is analyzed by the fluid delivery system. The fluid may then flow from the cartridge to a waste reservoir.

C. Microsieve

A microsieve-based detection system may be coupled to, positioned in, or positioned on a cartridge. The microsieve-based detection system may be integrated in the cartridge.

In some embodiments, a microsieve is selected depending on the analyte of interest. The microsieve may capture or retain matter in the sample (e.g., particles, cells, or other matter). Matter may be retained on a surface of the microsieve and/or in the microsieve. The microsieve may include a thin film or layer capable of separating one or more components from a liquid passing through the film or layer. The surface of a microsieve maybe hydrophilic to promote cell proliferation across the surface of the microsieve. A microsieve may have a variety of shapes including, but not limited to, square, rectangular, circular, oval, and/or irregularly shaped. In some embodiments, a microsieve includes openings (e.g., pores) that inhibit an analyte of interest from passing through the microsieve. A microsieve designed to capture substantially all of an analyte of interest may be selected depending on the analyte of interest.

In some embodiments, a microsieve is a monolithic microchip with a plurality of high-density holes. The monolithic microchip microsieve may be formed from materials including, but not limited to, glass, silica/germanium oxide doped silica, inorganic polymers, organic polymers, titanium, silicon, silicon nitride, and/or mixtures thereof. Organic polymers include, but are not limited to, PMMA, polycarbonate (PC) (e.g., NTJCLEOPORE® membrane\, Whatman, Florham Park, N.J.), and resins (e.g., DELRIN®, Du Pont, Wilmington, Del.). A microsieve formed of polymeric material may include pores of a selected range of dimensions, hi certain embodiments, a microsieve is an acrylic frit, hi some embodiments, a microsieve is formed of multiple layers (e.g., at least 2 layers, at least 3 layers, at least 4 layers, or at least 5 layers) of etchable and/or non-etchable glass, hi some embodiments, a microsieve is formed from an anti-reflective material and/or a material that does not reflect light in the ultraviolet-visible light range. In some embodiments, a microsieve includes one or more locking mechanisms to assist in securing placement of the microsieve in or on the cartridge or microsieve support.

Microsieves may have a thickness from about 0.001 mm to about 25 mm, from about 1 mm to about 20 mm, or from about 5 mm to 10 mm. In some embodiments, a thickness of the microsieve ranges from about 0.001 mm to about 2 mm. Microsieves may have a diameter from about 1 mm to 500 mm, from about 5 mm to about 100 mm, or from about 10 mm to about 50 mm.

Pores of a microsieve may have various dimensions (e.g., diameter and/or volume). In some embodiments, pores of the microsieve may have approximately the same dimensions, hi some embodiments, microsieve pores have a pore diameter ranging from about 0.0001 mm to about 1 mm; from about 0.0002 mm to about 0.5 mm; from about 0.002 mm to about 0.1 mm. The microsieve pores have, in some embodiments, a pore diameter of at most 0.005 mm or at most 0.01 mm.

Pores of the microsieve may be randomly arranged or arranged in a pattern (e.g., a hexagonal close-packed arrangement). Pores of the microsieve may occupy at least 10 percent, at least 30 percent, at least 50 percent, or at least 90 percent of the surface area of a microsieve. The pores may assist in selectively retaining matter in a sample and/or a fluid; including, for example, selected cell types like white blood cells.

In some embodiments, a microsieve is positioned from about 0.3 mm to about 0.5 mm below a top surface of the cartridge. In some embodiments, the microsieve includes a support, hi some embodiments, a microsieve is designed such that a microsieve support is not needed (e.g., utilizing a microsieve having a thickness of at least 5 mm). In some embodiments, one or more layers separate the microsieve and the microsieve support. The microsieve support may facilitate positioning of the microsieve in or on the cartridge.

A support assembly may be coupled to the microsieve support to allow the microsieve and microsieve support to withstand backpressures of at least 10 psi. The microsieve support may be selected to produce a predetermined backpressure. When backpressure is controlled, cells may be more uniformly distributed across a surface of a microsieve. Uniform distribution of cells across a microsieve surface may facilitate imaging of a region containing cells and/or analyte detection.

In some embodiments, a microsieve support includes open areas (e.g., pores or holes). Open areas in the microsieve support may have any shape, such as substantially square and/or substantially circular. The shape of the open areas in the microsieve support may be different than the shape of pores in the microsieve. Open areas of the microsieve support may be equal to or greater than the diameter of the pores of the microsieve. In some embodiments, a microsieve support has open areas with diameters ranging from about 0.0001 mm to about 1 mm, from about 0.0002 mm to about 0.5 mm, or from about 0.002 mm to about 0.1 mm. The open areas have, in some embodiments, diameters of at most 0.005 mm or at most 0.01 mm.

In a microsieve-based detection system, a fluid and/or sample in the detection region of the cartridge may be treated with a light. Interaction of the light with the fluid and/or sample may allow the analyte to be detected. Light from one or more light sources may shine on or in at least the detection region of a cartridge, such as the portion of the microsieve where the fluid and/or sample is retained. The light may allow a signal from the retained fluid and/or sample to be detected. When light shines on a microsieve surface, some of the light may be reflected. Areas proximate the detection region may also reflect some of the light that shines on a sample. Light reflecting from the microsieve surface and/or microsieve support may interfere with obtaining an accurate reading from the detector and so it may be advantageous to optically couple an anti-reflective material to the microsieve and/or the microsieve support.

In some embodiments, an anti-reflective material is optically coupled to the microsieve and/or the microsieve support. Alternatively, an anti-reflective material may be a coating on a surface of the microsieve and/or microsieve support. For example a black coating on a surface of the microsieve and/or microsieve support may act as an anti-reflective coating.

In certain embodiments, a portion of the microsieve and/or microsieve support may be made of an anti-reflective material. The anti-reflective material may be positioned above or below a microsieve. An anti-reflective material may inhibit the reflection of light applied to analytes retained in or on the microsieve. The anti-reflective material may absorb one or more wavelengths of light that are emitted by an analyte of interest. The anti-reflective material may improve the contrast of an image of at least a portion of the analyte retained in or on the microsieve by inhibiting reflection of light.

In some embodiments, materials that form the components of the cartridge control flow of fluids through the cartridge. In some embodiments, hydrophilic material is coupled to the microsieve and/or microsieve support. Alternatively, hydrophilic material may be a coating on a surface of a microsieve and/or microsieve support. In certain embodiments, a portion of the microsieve and/or microsieve support is made from hydrophilic material. Hydrophilic material may enhance flow of a fluid through the microsieve. Hydrophilic material may reduce the formation of air bubbles across the microsieve and microsieve support and/or inhibit nonspecific binding of analytes. Hydrophilic material may attract or have an affinity for aqueous fluids flowing through the microsieve. Hydrophilic material may be positioned downstream of the microsieve.

In some embodiments, hydrophobic material is positioned in or on the cartridge. Hydrophobic material may repel aqueous fluid away from surfaces of the cartridge and cause the fluid to flow towards the microsieve. For example, positioning a top member above the microsieve forms a cavity between the top member and the microsieve. Hydrophobic material may be coupled to the top member. The hydrophobic material may be a coating on a surface of the top member, and/or the hydrophobic material may form a portion of the top member. As an aqueous sample or fluid enters the cavity, it is repelled away from the hydrophobic top member and flows towards the microsieve.

D. Reagent Reservoir

In some embodiments, one or more reagents may be contained in a reservoir positioned on a cartridge. A reagent reservoir may include a blister pack, which may include one or more reagents in a sealed reservoir. A sealed reservoir may substantially contain reagents in the reservoir until needed. Pressure applied to a blister pack may break one or more surfaces of the blister pack such that reagent is released from the blister pack. In an embodiment, a blister of a blister pack may be formed of a first material and a second material, where a second material is configured to rupture or break prior to the first material when pressure is applied to the blister, hi an embodiment, a blister may include a first material configured not to break when pressure is applied to a blister and a second material configured to break when pressure is applied to a blister. A blister may be made of polyvinyl chloride (PVC); polyvinylidene chloride (PVDC); polyethylene (PE); polypropylene (PP); polyacrylonitrile (PAN); cyclic olefin copolymer (COC); fluoropolymer films; foil such as aluminum foil or plastic foil; and/or combinations thereof. A wall of a blister may be formed of layers of polypropylene, cyclic olefin copolymer. For example, a blister wall may be formed from a layer of cyclic olefin copolymer in between two layers of polypropylene. A wall of a blister may be formed of layers of polypropylene, cyclic olefin copolymer, and polyacrylonitrile. In an embodiment, a wall of a blister may be formed of layers of polyvinyl chloride, cyclic olefin copolymer, and polyvinylidene chloride.

E. Sample Collection and Processing

Collecting a sample includes taking a sample of blood from a subject using methods such as withdrawing blood from a needle inserted into the subject's blood vessel, withdrawing blood from a port inserted in a blood vessel of the subject, or puncturing the subject's skin with a sharp needle, lancet, finger-stick or heel-stick and collecting the subject's blood.

An instrument or system may be used to analyze one or more samples. A sample may include one or more analytes, cells, and/or bacteria. A sample may be collected for analysis with a sample collection device. The sample collection device may be external or internal to the instrument and may be interfaced with the analysis instrument. Depending on the type of measurement to be performed, a sample may be transported through one of two pathways by the sample collection device. In one application, a sample may be transported to an off-line sample-processing unit where the sample may be manipulated. The sample may then be transported to a disposable via a fluid delivery system. In another embodiment, a sample may be transported directly to a disposable cartridge by a sample collection device. The disposable cartridge, including the sample, may then be inserted into the instrument.

In an embodiment, a sample collection device may include a disposable pipette or capillary tube. A disposable pipette may contain, or may be coated with, one or more appropriate reagents to aid in visualization. For example, a stain may aid in visualization of particles and/or cells in a sample. A disposable pipette may also collect a precise sample volume. It may be desirable to incubate a sample prior to analysis. A sample may be incubated in a disposable tip before being drawn into an instrument. In one embodiment, after incubation, the sample may be delivered to the cartridge manually using the disposable pipette, hi another embodiment, a sample cartridge may include one or more appropriate reagents for incubation in the sample or reagent reservoir. In some embodiments, incubation may be performed within the sample cartridge using reagents from a sample or reagent reservoir. After the sample is incubated with one or more reagents, the fluid delivery system may deliver a buffer solution to the sample/reagent reservoir. Delivering a buffer solution to the sample/reagent reservoir may push the labeled sample to a microsieve in the cartridge for subsequent rinsing and sample analysis. After analysis of the sample is completed, the sample may be delivered to a waste reservoir. A waste reservoir may be positioned in the sample cartridge, internal or external to the instrument.

In an embodiment, a portion of a human body, such as a finger or heel, may be positioned proximate a sample reservoir of a cartridge. A portion of a human body may contact a portion of the sample reservoir. A sample reservoir may have a size that allows a predetermined volume of sample to be collected. A cartridge sample reservoir may include a sample pick-up pad. A sample pick-up pad may be a pad that absorbs and/or collects samples deposited on a surface of the sample pick-up pad. A sample pick-up pad may be made of an absorbent material. A sample pick-up pad may draw a sample from a portion of a human body in contact with the sample pickup pad to a sample reservoir. For example, a sample collection device may make a small incision in a portion of a human body. The portion of the human body may be brought proximate a sample pick-up pad. Blood from the small incision may flow onto the sample pick-up pad. Blood from the sample pick-up pad may then be delivered to the cartridge via a fluid delivery system. In an embodiment, a sample pick-up pad may include one or more anti-coagulants and/or reagents for sample labeling. A sample reservoir may include one or more anti-coagulants and/or reagents for sample labeling.

During analyte testing a sample may be introduced into an analyte detection device (e.g., a cartridge or lab-on-a-chip). A trigger parameter may be measured to determine when to introduce the binding agent/detectable label complex into the analyte detection device. Measurement of the trigger parameter may be continuous or may be initiated by a user. Alternatively, the detectable label may be introduced into the analyte detection device immediately after the sample is introduced.

In some embodiments, the trigger parameter may be the time elapsed since initiation of introducing the fluid into an analyte detection device at a controlled flow rate. For example, a binding agent/detectable label complex may be introduced 20 seconds after initiation of introducing the fluid sample into an analyte detection device at a flow rate of 1 milliliter per minute. In another embodiment, the trigger parameter may be the pressure drop across the microsieve. The pressure drop across the microsieve may be determined using a pressure transducer located on either side of the microsieve.

In some embodiments, the trigger parameter may be the autofluorescence of the analyte captured by the microsieve. A detector may be switched on until a predefined level of signal from the autofluorescence of the analyte has been reached. In still another embodiment, filtering software may be used to create a data map of the autofluorescence of the matter on the microsieve that excludes any pixels that contain color in a chosen spectral range. For example, the data map may be used to compute a value for particles that are autofluorescent only in the “pure green” portion of the visible spectrum.

F. Image Collection and Analysis

During use, a detectable label may cause emission of different wavelengths of light depending on the nature of the label. When the detecable label is analyzed with a camera, a user may be able to determine if a particular analyte is present based on the color or presence of emission at a given wavelength. For example, a green label may indicate the presence of an analyte of interest. Any other colored labels may not be of interest to a user. While a person may be able to discern between colors, it is desirable for a computer system to also be able to discern different colors from a sample. Many detectors can only discern specific colors when analyzing an image. For example, many CCD detectors can only discern red, blue and green colors. Thus, a CCD detector may not be able to discern the difference between a particle that emits both blue and green light and a particle that just emits green light, although the color difference may be apparent to a person using the system. To overcome this problem a method of subtracting out particles having the “wrong” color may be used.

Detectable labels may be detected by the presence or absence of label at a certain wavelength. Thus, either a black and white or a color CCD detector is useful in the systems and methods taught herein. Whenever colors are referred to herein, the presence or absence of the label at the appropriate wavelength rather than the color reported may be assessed and/or visualized. Thus, for example, a “yellow,” “green,” “blue” or “red” cell or label referred to herein may appear white using a black and white CCD detector.

A detector may be used to acquire an image of the analytes and other particulate matter captured on a microsieve. Cells may collect on a microsieve along with dust and other particulate matter and be captured in an image produced from a detector. The image acquired by the detector may be analyzed based on pre-established criteria. A positive result may indicate the presence of a cell. The. test criteria maybe based on a variety of characteristics of the image, including, but not limited to, the size, shape, aspect ratio, or color of a portion or portions of the image. Applying test criteria may allow cells to be distinguished from dust and other particulate matter. During analysis, the flow of sample through from a fluid delivery system may be continued.

In an embodiment, the system may include a computer system. A computer system may include one or more software applications executable to process a digital map of the image generated using a detector. For example, a software application available on the computer system may be used to compare the test image to a predefined optical fingerprint. Alternatively, a software application available on computer system may be used to determine if a count exceeds a pre-defined threshold limit.

The analysis may indicate that an analyte of interest is present in the sample. In an embodiment, user-defined threshold criteria may be established to indicate a probability that one or more specific cells are present on the microsieve. The criteria maybe based on one or more of a variety of characteristics of the image. In some embodiments, the criteria may be based on pixel or color fingerprints established in advance for specific cells. The characteristics that may be used include, but are not limited to, the size, shape, or color of portions of matter on the image, the aggregate area represented by the matter, or the total fluorescent intensity of the matter. In an embodiment, the system may implement an automated counting procedure developed for one or more cells.

In some embodiments, pixel analysis methods may be used in the analysis of an image of a fluid or captured matter. For example, pixel analysis may be used to discriminate microbes from dust and other particulate matter captured on a microsieve. Pixel analysis may include analyzing characteristics of an image to determine whether a cell is present in the imaged fluid.

Pixel analysis may be based on characteristics including, but not limited to, the size, shape, color, and intensity ratios of an image or portions of an image. As an example, the total area that emits light in an image maybe used to conduct analysis. As another example, the green fluorescent intensity of an image may be used to conduct analysis. In an embodiment, an “optical fingerprint” for a type of cell may be established for use in pixel analysis. In some embodiments, pixel analysis may be based on ratios between values, such as an aspect ratio of an element of matter captured on an image. In other embodiments, pixel analysis may be based on threshold values.

In some embodiments, pixels of an image that do not fall within a color range specified by a user may be discarded from the image, hi one embodiment, a fluid may be stained to cause a microbe of interest to emit light in only the green portion of the visible spectrum. By contrast, dust and other debris contained in the fluid may emit light in combinations of green, blue, and red portions of the visible spectrum in the presence of the stain. To isolate the portion of the image that represents only the microbe of interest, binary masks may be created to eliminate light emissions caused by non-microbial matter from the image.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for diagnosing severe combined immunodeficiency (SCID) in an infant comprising:
(a) passing a blood sample from an infant through a flow cell comprising a microsieve that separates lymphocytes from the blood sample, wherein the lymphocytes are contacted with a fluorescence-emitting lymphocyte marker that binds CD4 and a fluorescence-emitting lymphocyte marker that binds CD 8, in combination with one or more of (i) a fluorescence-emitting lymphocyte marker that binds CD2, (ii) a fluorescence-emitting lymphocyte marker that binds CD19, or (iii) a fluorescence-emitting lymphocyte marker that binds CD56, forming a lymphocyte/marker complex;
(b) exposing a lymphocyte-marker complex to light at a wavelength suitable for excitation of the fluorescence-emitting lymphocyte marker; and
(c) imaging fluorescence signals from the fluorescence-emitting lymphocyte marker to assess the number of lymphocytes in the sample; and
(d) transforming the number of lymphocytes into a lymphocyte ratio and comparing the ratio with a selected reference, wherein a lower amount of the lymphocytes as compared to a normal control or about the same amount of the lymphocytes as compared to a SCID control is indicative of SCID in said subject.

2. The method of claim 1, wherein the blood sample is a finger stick blood sample.

3. The method of claim 1, wherein the blood sample is a heel stick blood sample.

4. The method of claim 1, wherein the blood sample is a venipuncture sample.

5. The method of claim 1, wherein lymphocytes are T cells, B cells or nature killer cells.

6. The method of claim 1, wherein the fluorescence-emitting marker is coupled to a fluorescence-emitting particle.

7. The method of claim 1, wherein said fluorescence-emitting particle has a red spectra.

8. The method of claim 7, wherein said particle is Alexa 647.

9. The method of claim 1, wherein said fluorescence-emitting particle has a green spectra.

10. The method of claim 9, wherein said green label is Alexa 488.

11. The method of claim 1, wherein said imaging comprises using a CCD camera or a CMOS detector.

12. The method of claim 1, wherein the lymphocyte ratio comprises the amount of a specific lymphocyte compared to the amount of total lymphocytes.

13. The method of claim 1, wherein the lymphocyte ratio comprises the amount of a specific lymphocyte in a specific volume.

14. The method of claim 1, wherein the selected reference is a threshold value.
